(19) United States
(12) Patent Application Publication
(10) Pub. No.: US 2026/0345177 A1
(43) Pub. Date: Nov. 5, 2026

(54) LATERAL MASS DRILL GUIDE SYSTEM

(71) Applicant: Providence Medical Technology, Inc., Pleasanton, CA (US)

(72) Inventors: Nicholas Domek, Sacramento, CA (US); Edward Liou, Danville, CA (US); Christopher U. Phan, Dublin, CA (US); Bruce M. McCormack, San Francisco, CA (US); Jamieson Glenn, Cardiff, CA (US)

(21) Appl. No.: 19/119,938

(22) PCT Filed: Oct. 10, 2023
(86) PCT No.: PCT/US2023/034784

§ 371 Date: Apr. 10, 2025

Related U.S. Application Data
(60) Provisional application No. 63/415,096, filed on Oct. 11, 2022.

(51) Int. Cl. A61B 17/17

(52) U.S. Cl. CPC A61B 17/1735; A61B 17/1757

(57) ABSTRACT

Disclosed herein is a drill guide system. The system includes an access chisel and a drill guide. The drill guide includes a cannulated body portion having a first lumen comprising a first central longitudinal axis, and a second lumen comprising a second central longitudinal axis. The drill guide also includes a proximal portion including a handle portion. The handle portion includes a first through hole to provide access to the first lumen, and a second through hole to provide access to the second lumen. The handle portion also includes a depth stop mechanism. The depth stop mechanism includes one or more slots defined in the handle portion and a depth stop bushing. The depth stop bushing includes a tubular body, a knob and a shaft connecting the knob and the tubular body. The shaft has a shaft protrusion extending a length of the shaft.

### CROSS-REFERENCE TO RELATED APPLICATION

This application claims the benefit of U.S. Provisional Application No. 63/415,096, filed Oct. 11, 2022, the disclosure of which is hereby incorporated by reference in its entirety.

### TECHNICAL FIELD

This disclosure is directed to medical devices, systems and methods. More specifically, this disclosure is directed to devices, systems and methods related to fusion procedures involving a lateral mass drill guide.

### BACKGROUND

Chronic back problems are one of the most common causes of pain and disability in the United States and other developed countries. Adverse spinal conditions may be characteristic of age. Conditions such as Degenerative Disc Disease (DDD) and spinal stenosis can result in a reduction of foraminal area (i.e., the available space for the passage of nerves and blood vessels), which may compress nerve roots and cause radicular pain. Spinal fusion, in which two adjacent vertebrae are fused together using plates, screws and other implants, is often performed to increase space (“spinal distraction”) and reduce motion between adjacent vertebrae, thereby preventing impingement of the spinal cord and nerve roots passing through openings in the vertebral column.

Posterior cervical fixation may use lateral mass screws and rods. A user (surgeon) will drill a hole in the lateral mass at a particular angle, depth and diameter. A well-known drilling technique is called the Magerl technique. In this procedure, the surgeon will manually drill a hole parallel to the adjacent facet joints with a medial-to-lateral trajectory of approximately 25°. In this open direct-visualization surgical technique, most surgeons will use a small high-speed burr to penetrate the cortical bone layer at the desired starting position, typically centered on the lateral mass or slightly superomedial to the center. A drill guide is placed over the starting position and a manual or high-speed drill is used to drill the starting hole to the proper depth. The hole can be tapped prior to inserting a lateral mass screw, or a self-tapping screw can be used. Unfortunately, this and other techniques are relatively invasive, high-risk, require increased surgery time and necessitate difficult recovery and rehabilitation.

Accordingly, a need exists for new, less invasive devices and methods for processes that aid vertebral fusion and require less surgery time.

### SUMMARY

The present disclosure describes devices, systems and associated methods related to a drill guide.

Disclosed herein is a drill guide system that may be anchored to a cervical facet joint and used to drill a cervical lateral mass. The system includes an access chisel and a drill guide. The drill guide includes a cannulated body portion having a first lumen comprising a first central longitudinal axis, and a second lumen comprising a second central longitudinal axis. The drill guide also includes a proximal portion including a handle portion. The handle portion includes a first through hole to provide access to the first lumen, and a second through hole to provide access to the second lumen. The handle portion also includes a depth stop mechanism. The depth stop mechanism includes one or more slots defined in the handle portion and a depth stop bushing. The depth stop bushing includes a tubular body, a knob and a shaft connecting the knob and the tubular body. The shaft has a shaft protrusion extending a length of the shaft.

In some embodiments, each of the one or more slots includes a bottom portion and a slot protrusion extending from a side proximate to a top of the bottom portion. The slot protrusion is configured to extend or retract when pressed with the shaft protrusion to lock or release the depth stop bushing in a respective slot. In some aspects, each of the one or more slots is U-shaped. In some aspects, the first and second central longitudinal axes are distinct and parallel. In some aspects, the handle portion further includes a mallet surface extending from a side of the handle portion. In some aspects, the first lumen is configured to receive the access chisel and the second lumen is configured to receive a drill. In some aspects, the distal portion of the second lumen further comprises serrated teeth configured for anchoring the drill guide into the cortical bone of the lateral mass. In some aspects, the drill guide also includes a ring feature configured to join the two lumens together proximate the distal portion of the drill guide. In some aspects, the access chisel is a facet access chisel and the drill guide is a lateral mass drill guide.

Additionally or alternatively, the drill guide system includes a drill guide burr. The drill guide burr includes an elongated body with a proximal portion opposite a distal portion. The proximal portion includes a positive stop feature and a quick connect feature configured to receive a handle. The distal portion includes a rounded cross-cut burr tip configured to create a divot in a lateral mass. The elongated body of the drill guide burr has a length, the proximal portion has a first diameter and the distal portion has a second diameter and the first diameter is greater than the second diameter.

Additionally or alternatively, the drill guide system includes a drill. The drill includes an elongated body with a proximal portion opposite a distal portion. The proximal portion includes a positive stop feature and a quick connect feature configured to receive a handle. The distal portion includes a short flute drill profile and an awl tip. The elongated body of the drill has a length, the proximal portion has a first diameter, the distal portion has a second diameter and the first diameter is greater than the second diameter.

In some aspects, the access chisel includes an elongated body having a proximal portion and a distal portion, a chamfered or beveled end feature positioned at the distal portion of the body and configured for insertion at a facet joint, an opening or window defined in the distal portion, and a stop feature defined in the distal portion and configured to abut a posterior edge or portion of a facet joint. Optionally, the distal portion of the access chisel may include a raised or protruding feature. The raised or protruding feature is a plurality of spikes or teeth. The opening or window is a visualization window configured for visualization of a superior or inferior lateral mass. In some aspects, the proximal portion has a first height and the distal portion at the tip has a second height and the first height is greater than the second height. In some aspects, the facet joint is a cervical facet joint.

Disclosed herein is a drill guide. In some aspects, the drill guide includes a cannulated body portion having a first lumen comprising a first central longitudinal axis, and a second lumen comprising a second central longitudinal axis. The drill guide also includes a proximal portion including a handle portion. The handle portion includes a first through hole to provide access to the first lumen, and a second through hole to provide access to the second lumen. The handle portion also includes a depth stop mechanism. The depth stop mechanism includes one or more slots defined in the handle portion and a depth stop bushing. The depth stop bushing includes a tubular body, a knob and a shaft connecting the knob and the tubular body. The shaft has a shaft protrusion extending a length of the shaft.

In some embodiments, each of the one or more slots includes a bottom portion and a slot protrusion extending from a side proximate to a top of the bottom portion. The slot protrusion is configured to extend or retract when pressed with the shaft protrusion to lock or release the depth stop bushing in a respective slot. In some aspects, each of the one or more slots is U-shaped. In some aspects, the first and second central longitudinal axes are distinct and parallel. In some aspects, the handle portion further includes a mallet surface extending from a side of the handle portion. In some aspects, the first lumen is configured to receive the access chisel and the second lumen is configured to receive a drill. In some aspects, the distal portion of the second lumen further comprises serrated teeth configured for anchoring the drill guide into the cortical bone of the lateral mass. In some aspects, the drill guide also includes a ring feature configured to join the two lumens together proximate the distal portion of the drill guide. In some aspects, the drill guide is a lateral mass drill guide.

An access chisel is disclosed herein. In some aspects, the access chisel includes an elongated body having a proximal portion and a distal portion, a chamfered or beveled end feature positioned at the distal portion of the body and configured for insertion at a facet joint, an opening or window defined in the distal portion, and a stop feature defined in the distal portion and configured to abut a posterior edge or portion of a facet joint. Optionally, the distal portion of the access chisel may include a raised or protruding feature. The raised or protruding feature is a plurality of spikes or teeth. The opening or window is a visualization window configured for visualization of a superior or inferior lateral mass. In some aspects, the proximal portion has a first height and the distal portion at the tip has a second height and the first height is greater than the second height. In some aspects, the facet joint is a cervical facet joint. In some aspects, the access chisel is a facet access chisel.

### BRIEF DESCRIPTION OF THE DRAWINGS

FIG. 1 is a lateral view of the C5 and C6 (cervical) vertebrae.

FIGS. 2A and 2B illustrate a drill guide system according to the present disclosure.

FIGS. 3A-3C illustrate an access chisel of the drill guide system of FIGS. 2A and 2B.

FIGS. 4A-4E illustrate a drill guide of the drill guide system of FIGS. 2A and 2B.

FIGS. 4F-1 through 4G-3 depict portions of a depth stop mechanism of the drill guide system of FIGS. 2A and 2B.

FIG. 5 depicts a drill guide burr of the drill guide system of FIGS. 2A and 2B.

FIG. 6 depicts a drill of the drill guide system of FIGS. 2A and 2B.

FIG. 7A-1 through FIG. 7H-3 depicts a method of using the drill guide system of FIGS. 2A and 2B.

### DETAILED DESCRIPTION

The following description of certain embodiments is merely exemplary in nature and is in no way intended to limit the invention or its applications or uses. In the following detailed description of embodiments of the present systems and methods, reference is made to the accompanying drawings which form a part hereof, and which are shown by way of illustration specific embodiments in which the described systems and methods may be practiced. These embodiments are described in sufficient detail to enable those skilled in the art to practice presently disclosed systems and methods, and it is to be understood that other embodiments may be utilized and that structural and logical changes may be made without departing from the spirit and scope of the present system. Moreover, for the purpose of clarity, detailed descriptions of certain features will not be discussed when they would be apparent to those with skill in the art so as not to obscure the description of the present system. The following detailed description is therefore not to be taken in a limiting sense, and the scope of the present system is defined only by the appended claims.

As used herein, “vertebrae” may refer to cervical or lumbar vertebrae unless otherwise indicated. Embodiments are not limited to cervical or lumbar vertebrae, however, and may encompass other vertebrae or groups of vertebrae along the spine of a subject.

As used herein, “subject” may refer to a human (e.g., a patient) afflicted with a spinal condition, e.g., spinal stenosis, injury or disease, which may be degenerative. The condition may impact the integrity and/or positioning of one or more vertebrae, which may cause chronic pain and instability.

The terms “distal” and “proximal” are used to refer to a position or direction relative to the treating clinician, such as a surgeon. “Distal” and “distally” refer to a position that is distant from, or in a direction away from, the treating clinician. “Proximal” and “proximally” refer to a position that is near, or in a direction toward, the treating clinician. The terms “posterior” and “anterior” refer to the back and front, respectively, of the body of a subject.

As used herein, “decortication” refers to the process by which an outer surface of a bone is roughened or removed and the underlying cancellous bone exposed. Relative to the outer bone surface, the cancellous bone more effectively retains deposited graft material, thereby promoting healing and bone growth after surgery.

For the following defined terms, certain definitions shall be applied unless a different definition is given elsewhere in this disclosure. The terms “a,” “an,” and “the” are used to include one or more than one, independent of any other instances or usages of “at least one” or “one or more.” The term “or” is used to refer to a nonexclusive or, such that “A or B” includes “A but not B,” “B but not A,” and “A and B.” All numeric values are assumed to be modified by the term “about,” whether or not explicitly indicated. The term “about” generally refers to a range of numbers that one of skill in the art would consider equivalent to the recited value (e.g., having the same function or result). In many instances, the term “about” can include numbers that are rounded to the nearest significant figure. The recitation of numerical ranges by endpoints includes all numbers and sub-ranges within and bounding that range (e.g., 1 to 4 includes 1, 1.5, 1.75, 2, 2.3, 2.6, 2.9, etc. and 1 to 1.5, 1 to 2, 1 to 3, 2 to 3.5, 2 to 4, 3 to 4, etc.).

Spinal stenosis reflects a narrowing of one or more areas of the spine, often in the upper or lower back. This narrowing can put pressure on the spinal cord or on the nerves that branch out from the compressed areas. Individual vertebrae of the spine are positioned relative to each other, and their separation is maintained by discs separating main vertebral bodies and by capsules positioned within facet joints. The discs and capsules are separated from the bone of their respective joints by cartilage. Spinal stenosis is often indicative of degeneration of a disc, a capsule, or the cartilage in a joint, which leads to a compression of the joints and the narrowing mentioned.

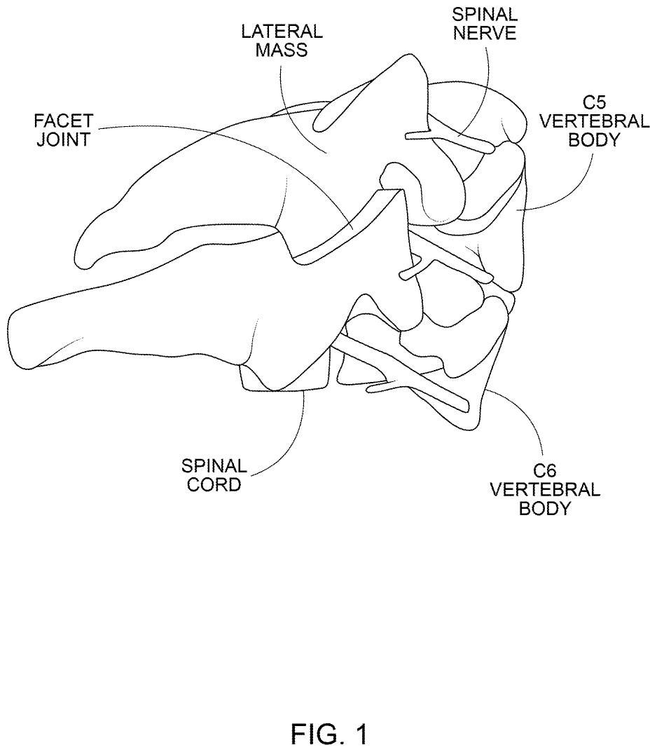

Options for distracting two adjacent vertebrae of a spine, such as the cervical vertebrae shown in FIG. 1, in an effort to ameliorate spinal stenosis, are varied and may include the use of implants, or cages and screws. In many cases, it may be possible to insert a facet joint implant into a facet joint by itself and, due to the design of the implant, do nothing further to secure the implant within the joint. In other words, the shape, size, surface features and overall configuration of the implant may cause it to remain securely within the facet joint without further attachment devices required. In some cases however, and in general for overall safety of a facet joint distraction procedure, it may be advantageous to use one or more additional devices, such as screws, to help secure the facet joint implant to one or both of the adjacent vertebrae that form the joint with a screw.

Still further, it may be advantageous to provide additional stabilization to the fusion site with the use of a pedicle screw and/or a lateral mass screw as a supplement to spinal fusion surgery. A pedicle screw or lateral mass screw are types of bone screws designed for insertion into the pedicle or lateral mass of a vertebra, respectively. The screws are inserted in adjacent vertebrae (e.g., consecutive spine segments such as C5 and C6) and then connected via a rod to prevent motion at those segments that are being fused. In this way, the screws act as anchor points for the rod and provide additional stability to fusion site to promote better fusion. Post-fusion, the rods and screws can be removed.

Accessing the cervical spine via a posterior approach utilizes minimally invasive or less invasive techniques. Aspects described below generally include an access tool and a guide tool, through which or along which one or more spinal fixation devices may be advanced. In addition, the access tool may also have a rasp tip to serve as a decortication tool. The decortication tool is used for preparing the bone surface prior to insertion of the spinal fixation device.

Aspects disclosed herein also include a drill guide system that can be anchored to a facet joint, such as a cervical facet joint. The drill guide may include a fixed or adjustable depth stop to guide a drill during a drilling procedure. The disclosed drill guide also provides angulation control to provide superior-inferior and/or medial-lateral angulation control to prevent violation of the vertebral artery or nerve root. Using the access chisel as an anchor, the drill is parallel to the facet joint and the guide provides surgeon control over the angulation of the drill entry point. The drill is less likely to slip off or walk off the lateral mass while drilling. These advantages reduce surgery time and anesthesia exposure for the patient.

In use, the surgeon advances the access tool into the facet joint through a minimally invasive or less invasive incision. Once anchored into place, this access tool provides a fixed point deep in the spine that is then used as a marker to advance drills, awls, plates, rods and screws, and other instruments to the cervical spine from a posterior approach without direct visualization. Such an approach with the disclosed devices prevents instruments from slipping off the spine or drills catching soft tissue and skidding out of control. In addition, the cervical facet has a fixed anatomical relationship to lateral mass bone consistent in most, if not all, patients. Instruments can be advanced over, along or about the access tool to reliable landmarks on or at the lateral mass without direct visualization. For example, to aid in spinal fixation, lateral mass screws or pedicle screws may be inserted with the help of a guide tool.

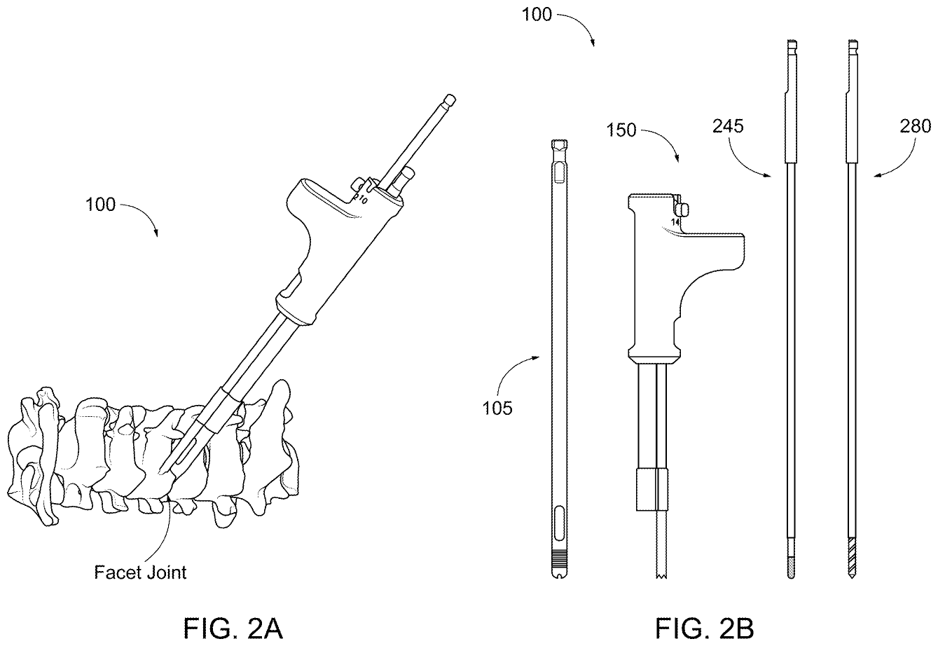

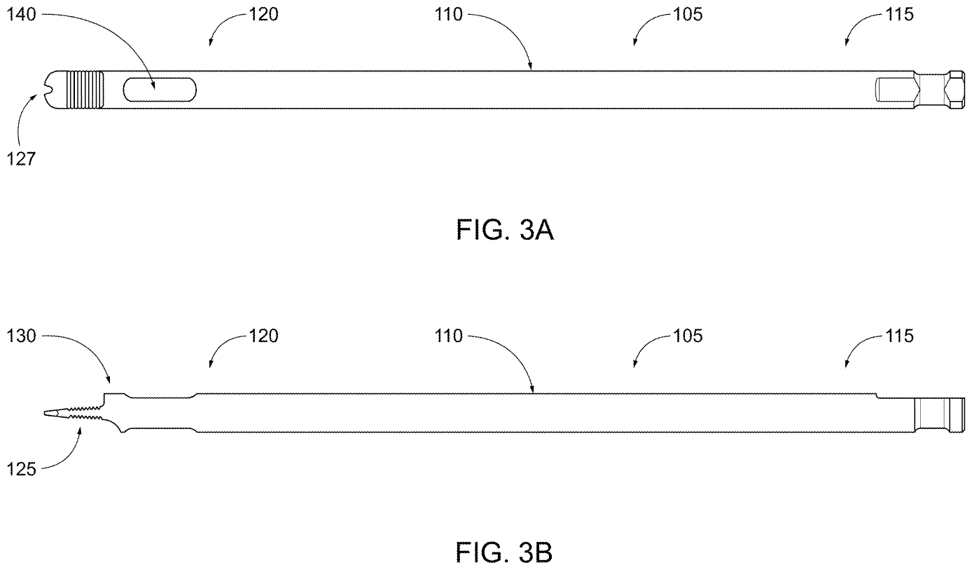

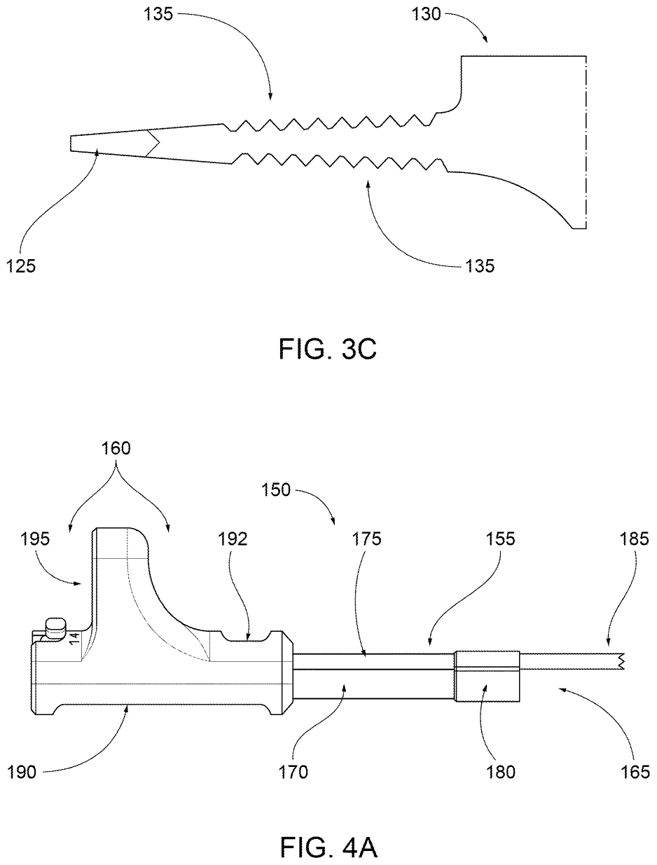

Turning now to the figures, FIG. 2A illustrates the facet anchored drill guide system in use and FIG. 2B illustrates various tools or instruments that may be included in the facet anchored drill guide system 100. As can be understood from FIGS. 2B and 3A-3C, the access chisel or device 105 includes a body 110 which may be an elongated tubular body having a proximal portion 115 and a distal portion 120. The distal portion 120 further includes a chamfered or beveled end feature 125 with a split point tip 127 positioned at an end of the distal portion of the access device. The distal portion 120 further includes a stop feature 130 configured to abut the posterior edge of the facet joint (see e.g, FIG. 2A). The distal portion 120 also includes a raised or protruding feature 135, such as a plurality of spikes or teeth. The distal portion 120 further includes an opening or window 140 to provide visualization of the interior facet joint. The opening or window may have a generally oval shape, but other shapes such as rectangular with rounded edges or a rounded shape can also be used. In use, the raised or protruding feature 135 engages the facet joint to anchor the device 105 into the facet joint and the stop feature 130 abuts the posterior edge of the facet joint and the raised or protruding feature 135 engages the joint to anchor the device. Advantageously, the split-point tip eases insertion, the positive stop prevents over-insertion, the serrated teeth provide anchoring and decortication, and the window provides visualization of the inferior facet joint.

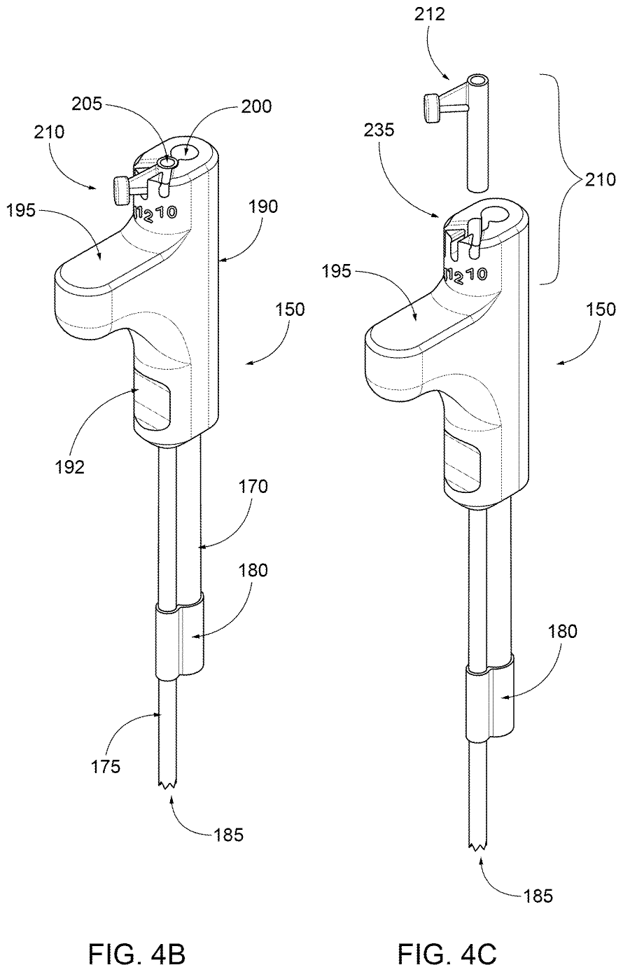

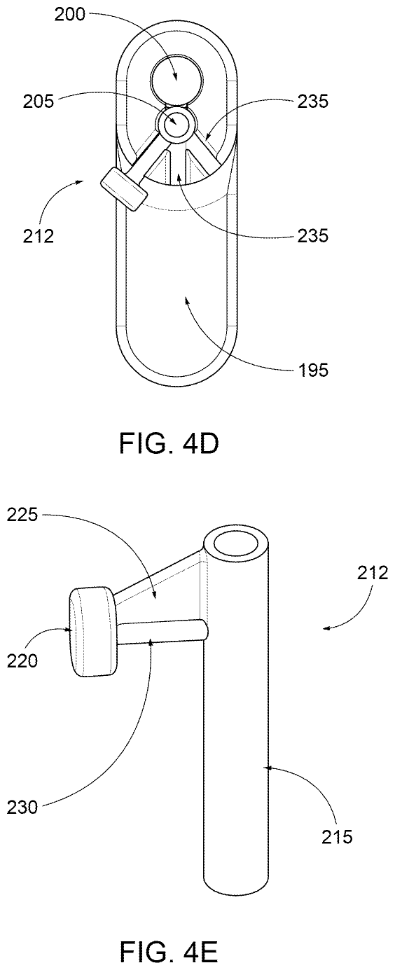

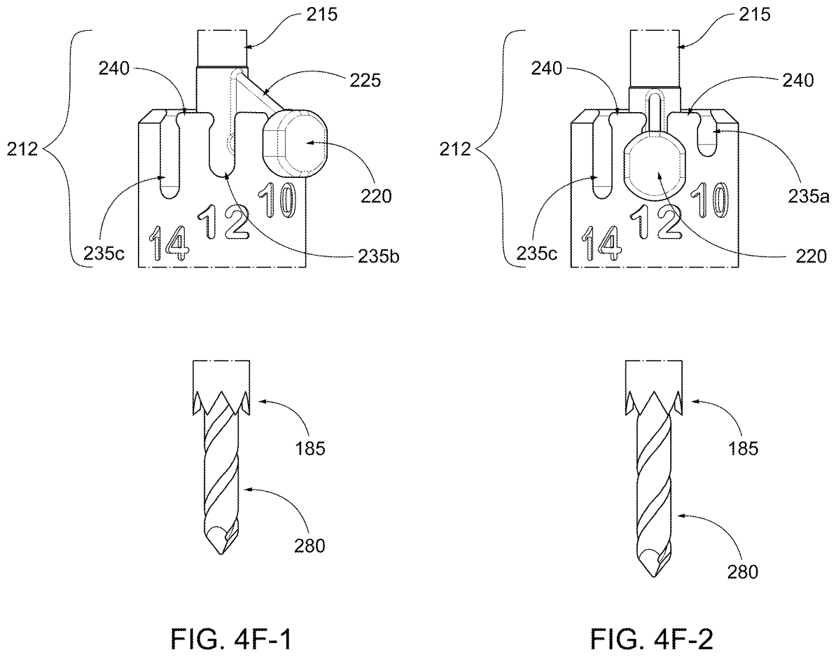

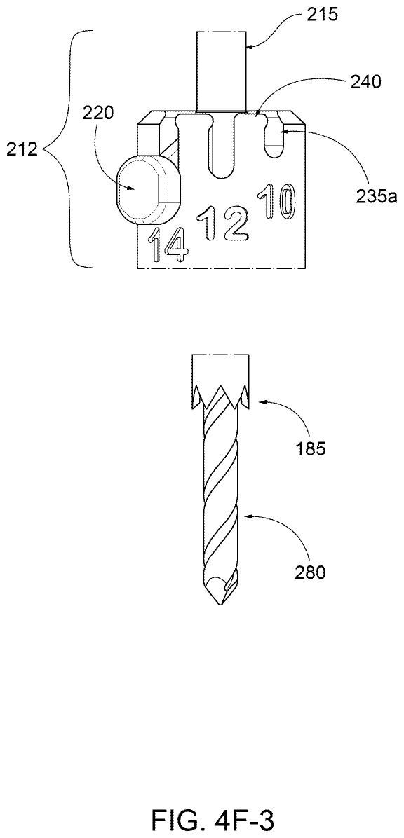

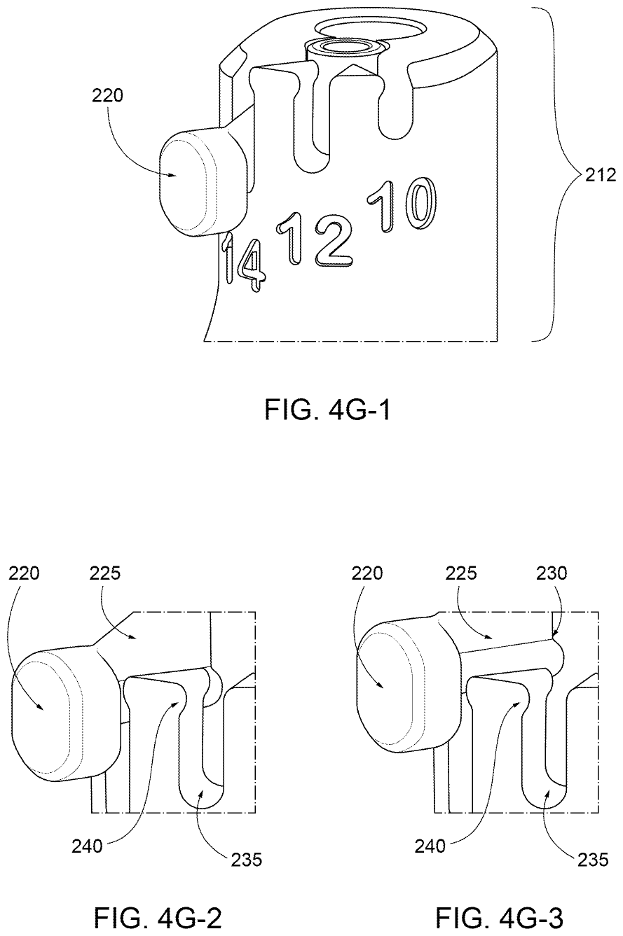

As shown in FIG. 2B, the facet anchored drill guide system 100 may also include a drill guide 150. As depicted in FIGS. 4A-4E and others, the drill guide 150 includes a cannulated body portion 155, a proximal portion 160 and a distal portion 165. The cannulated body portion may have two lumens or cannulas, the first lumen or cannula 170 having a first central longitudinal axis and the second lumen or cannula 175 having a second central longitudinal axis. The first and second central longitudinal axes are distinct and parallel. The first lumen 170 is configured to receive the access chisel 105 and the second lumen 175 is configured to receive a drill. The two lumens may be joined together by a ring feature 180 proximate the distal portion of the drill guide. The distal portion of the second lumen also includes serrated teeth 185 that may be used for anchoring the drill guide into the cortical bone of the lateral mass. The teeth hinder or prevent the drill guide from rotating about the access chisel.

The proximal portion 160 of the drill guide 150 includes a handle portion 190 with a grip portion 192. Extending from a side of the handle 190 is a mallet surface 195 which may be used if or when the drill guide 150 needs to be malleted into place to anchor the drill guide into the lateral mass. The handle portion 190 also includes a first through hole or opening 200 to the first lumen 170 for the access chisel 105 and a second through hole or opening 205 for the second lumen 175 to receive a drill.

As can be understood from FIGS. 4B to 4G-3, the handle portion 190 of the drill guide 150 further includes a depth stop mechanism 210. The depth stop mechanism 210 includes a depth stop bushing 212 including a tubular body 215, a knob 220 and a flange or shaft 225 connecting the knob 220 and the tubular body 215. The flange or shaft 225 includes a shaft protrusion 230 extending the length of the shaft. The tubular body 215 is received in the second lumen 175 of the handle portion 190 and is configured to accept a drill. The depth stop mechanism 210 further includes one or more slots 235 defined in the handle portion 190 that define the depth of the depth stop. That is, and as shown in FIGS. 4F-1, 4F-2, 4F-3 and 4G-1, and others, in one embodiment, there are three depth stop positions, labeled “10”, “12” and “14”. In use, the surgeon can set the drill depth by moving the depth stop bushing 212 into the radial slot 235 of the desired depth. Thus, in a first slot 235a, the drill protrudes from the distal tip at a first depth (see FIG. 4F-1). In a second slot 235b, the drill protrudes from the distal tip at a second depth (see FIG. 4F-2). In a third slot 235c, the drill protrudes from the distal tip at a third depth (see FIG. 4F-3).

In one embodiment, and as shown in FIGS. 4F and 4G, and others, the one or more slots 235 may be U-shaped and includes a radial cantilevered snap or locking mechanism or slot protrusion 240 extending from the sides at the top or proximal portion of the slot 235. Other shaped slots may also be used, such as V-shaped slots. In use, the surgeon can apply pressure to release or lock the depth stop bushing into the desired position. The proximal portion is made of an engineering plastic that flexes when the knob is pressed over the snap/lock feature (slot protrusion) thereby locking or unlocking the depth stop. As can be understood from the figures, including FIGS. 4G-2 and 4G-3, pressing down locks the depth stop bushing into the slot and pulling upwards unlocks the depth stop bushing from the slot.

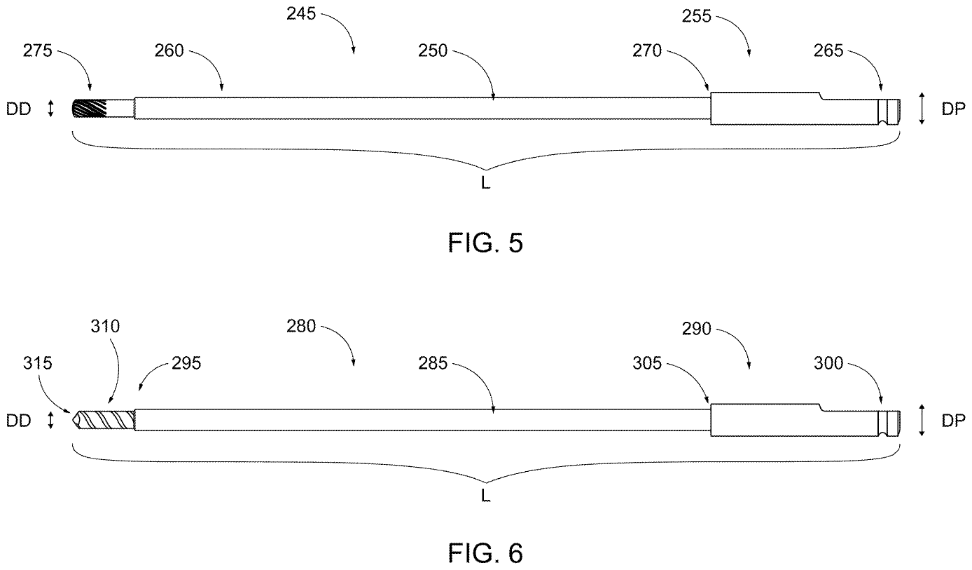

As can be understood from FIGS. 2A and 2B, the drill guide system 100 may further include a drill guide burr 245. As shown in FIG. 5, the drill guide burr 245 includes an elongated body 250 with a proximal portion 255 opposite a distal portion 260. The proximal portion 255 includes a quick connect feature 265, such as an a/o quick connect feature, that can accept a handle (not shown). The proximal portion 255 also includes a positive stop feature 270. The distal portion 260 includes a tip 275, which may be a rounded cross-cut burr tip, configured to create a divot in the lateral mass. This divot will be the entry point for the drill. The elongated body 250 has a length L and the diameter of the proximal portion DP is greater than the diameter of the distal portion DD.

As can be understood from FIGS. 2A and 2B, the drill guide system 100 may further include a drill 280. As can be understood from FIG. 6, the drill 280 includes an elongated body 285 with a proximal portion 290 opposite a distal portion 295. The proximal portion 290 includes a quick connect feature 300, such as an a/o quick connect feature, that can accept a handle (not shown). The proximal portion 290 also includes a positive stop feature 305. The distal portion 295 includes a short flute drill profile 310 and an awl tip 315. Other drill profiles and tip configurations may also be used. The elongated body 285 has a length L and the diameter of the proximal portion DP is greater than the diameter of the distal portion DD.

The drill guide system 100 is used to drill one or more holes in the lateral mass for accurate placement of a fixation assembly, such as a rod and screw set. Turning now to FIGS. 7A-1 through FIG. 7H-3, a method of using the disclosed drill guide system 100 is depicted. The method may include additional steps or fewer steps and the steps may be performed in a different order.

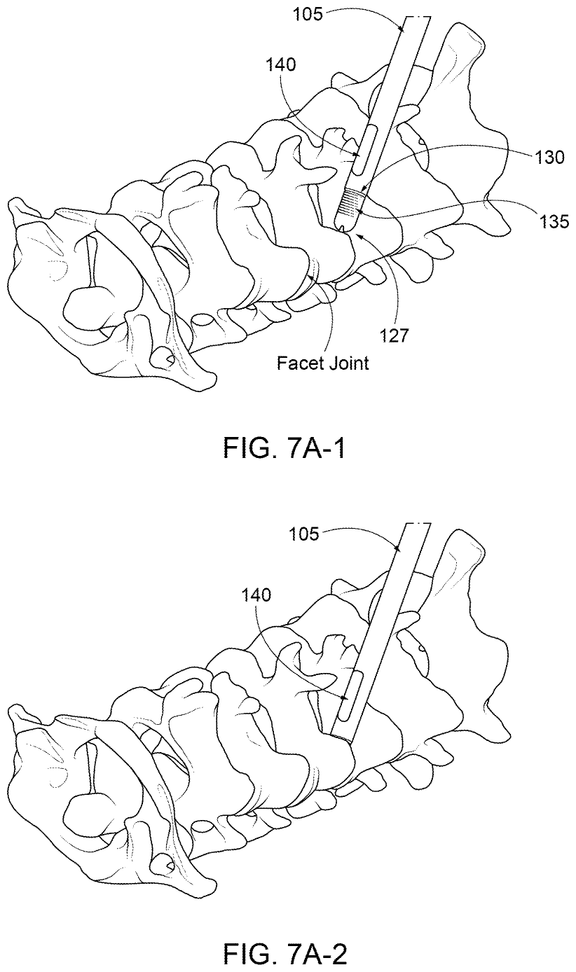

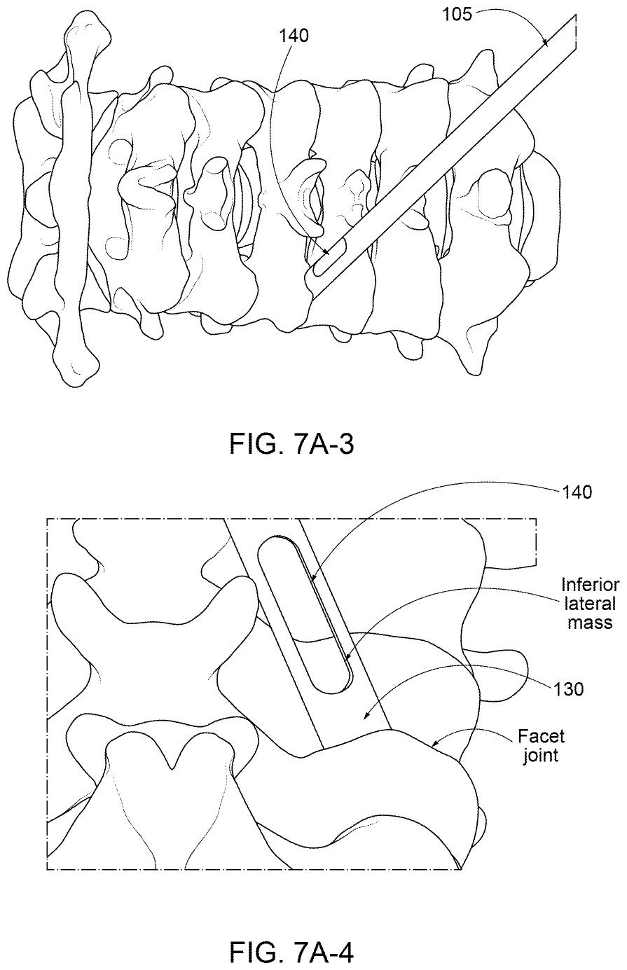

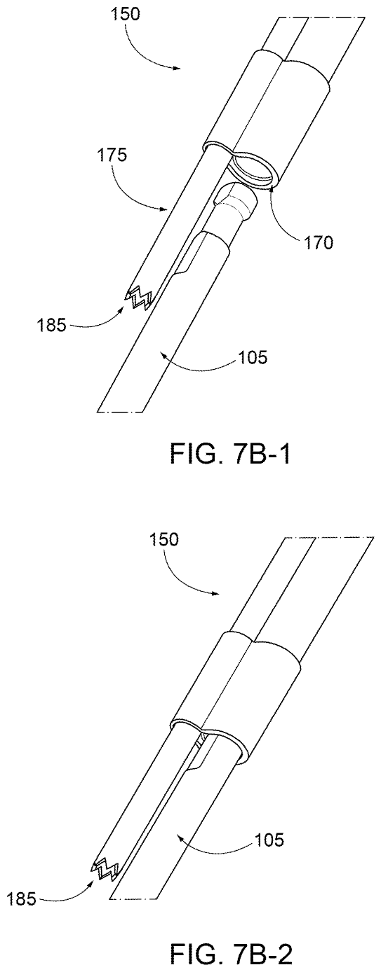

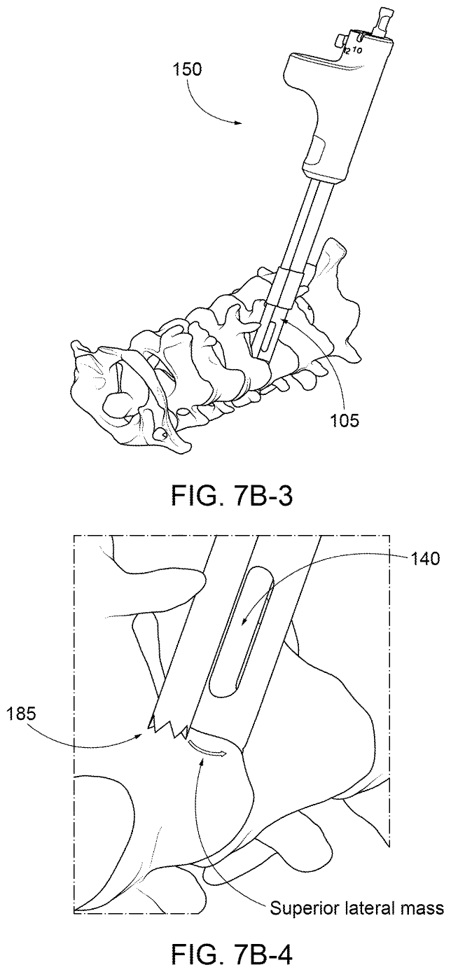

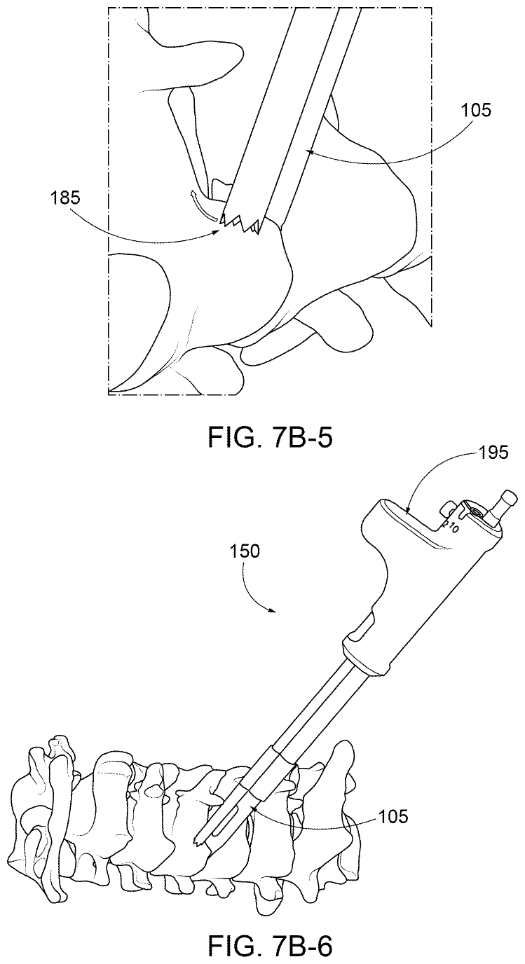

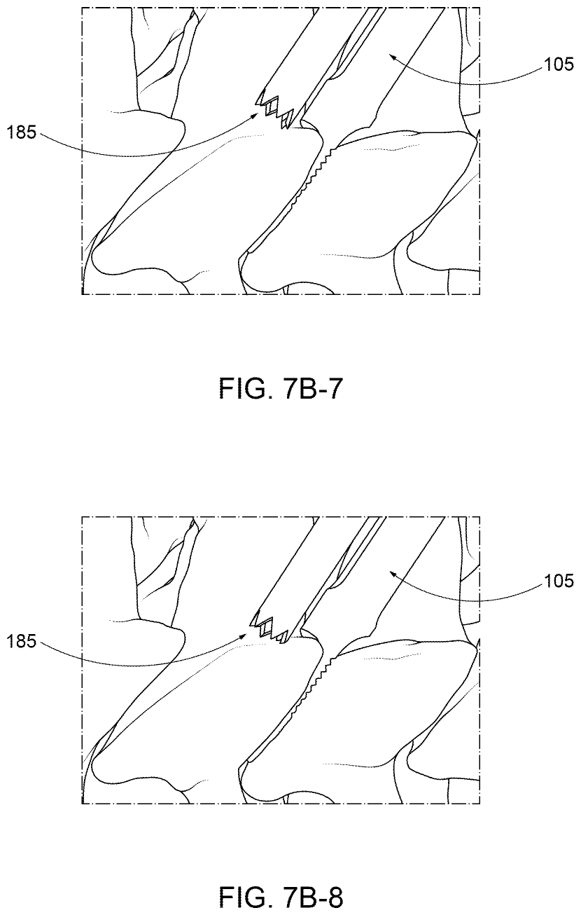

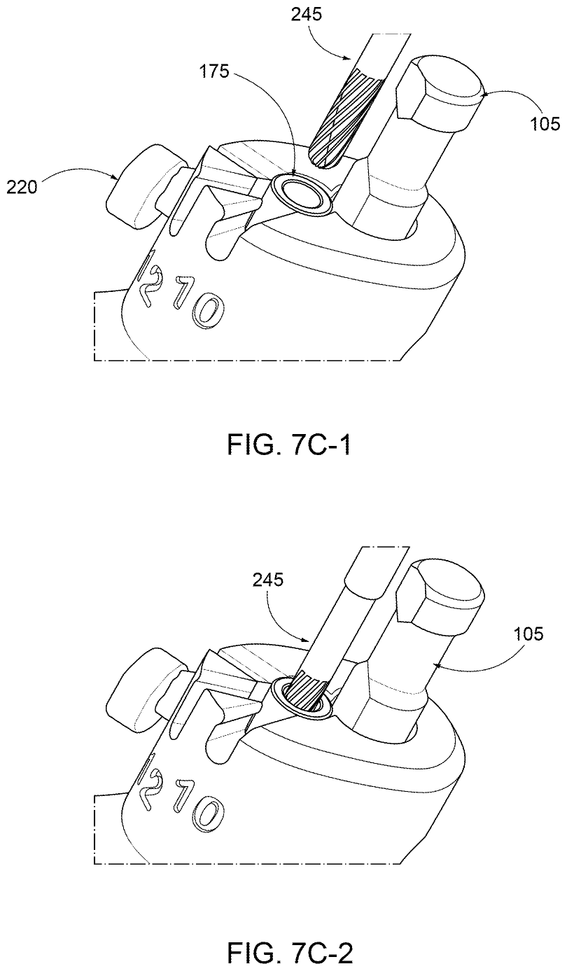

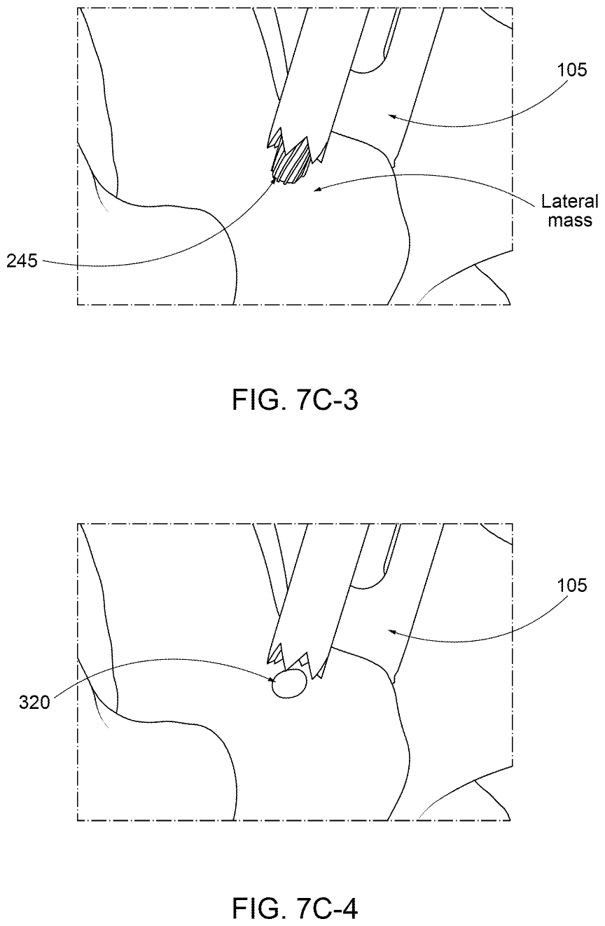

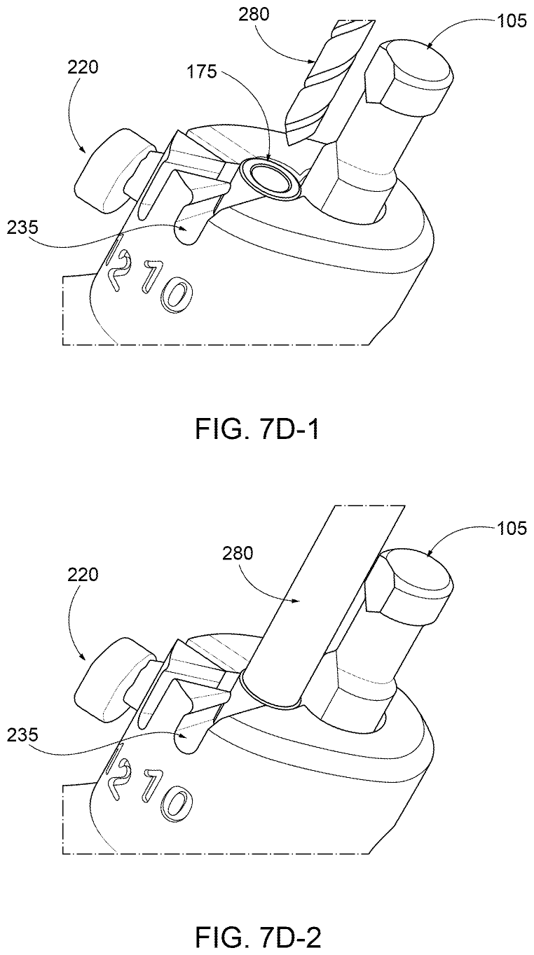

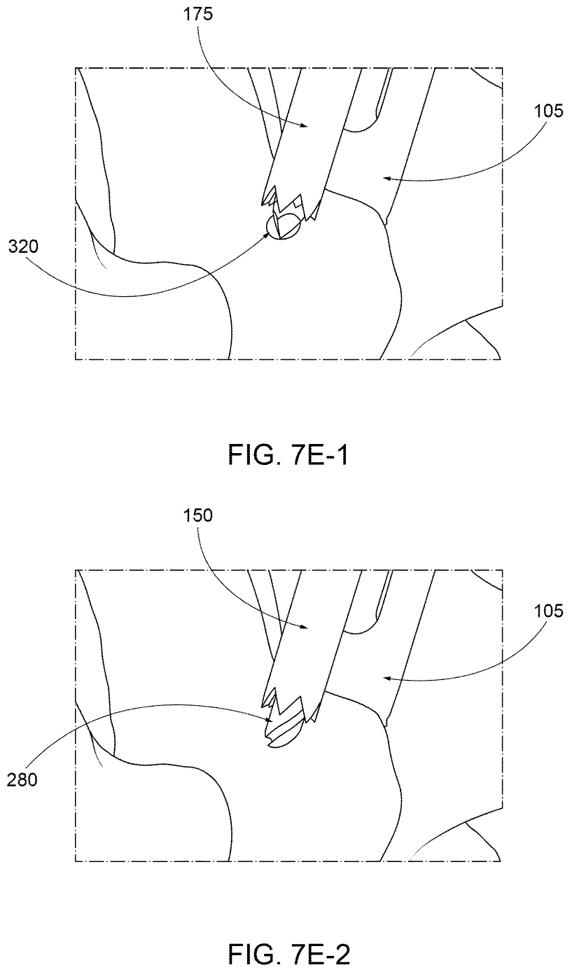

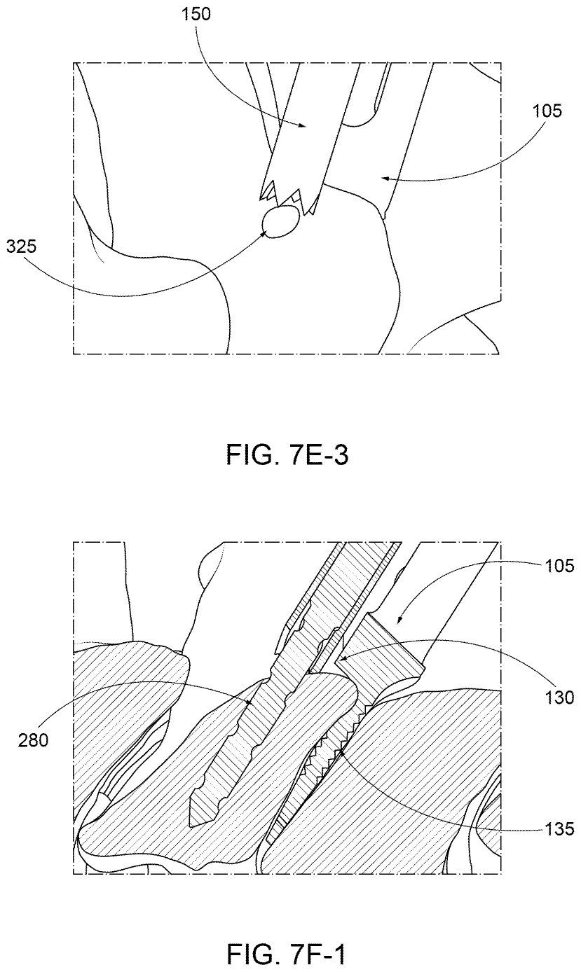

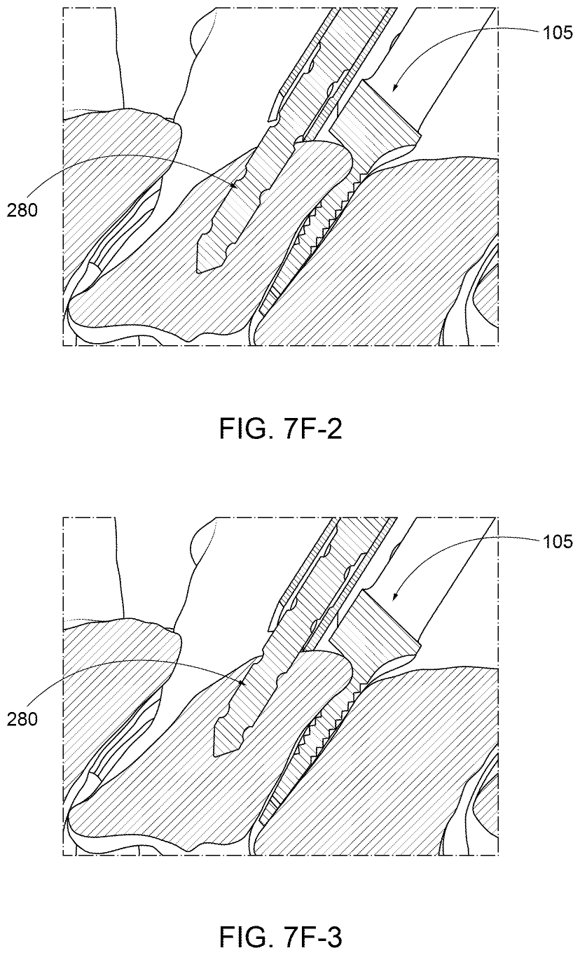

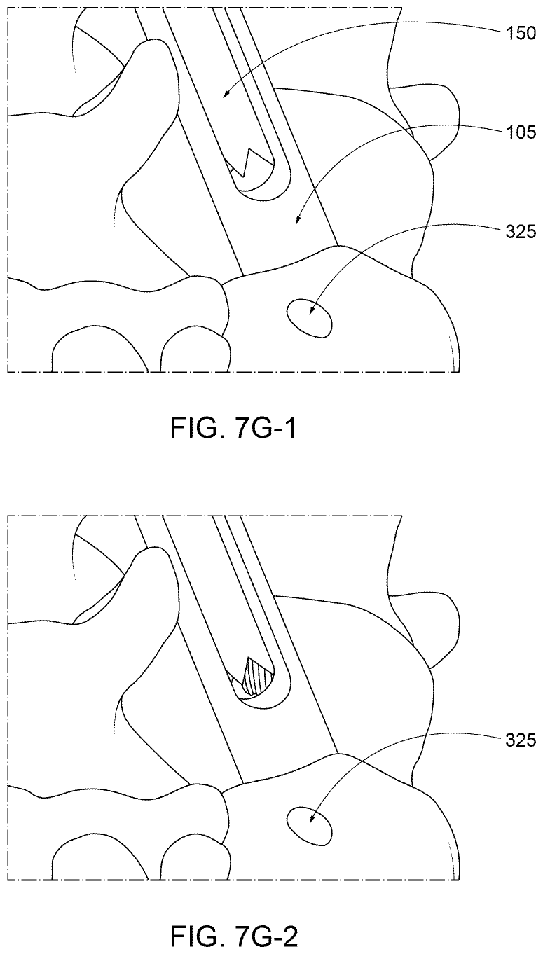

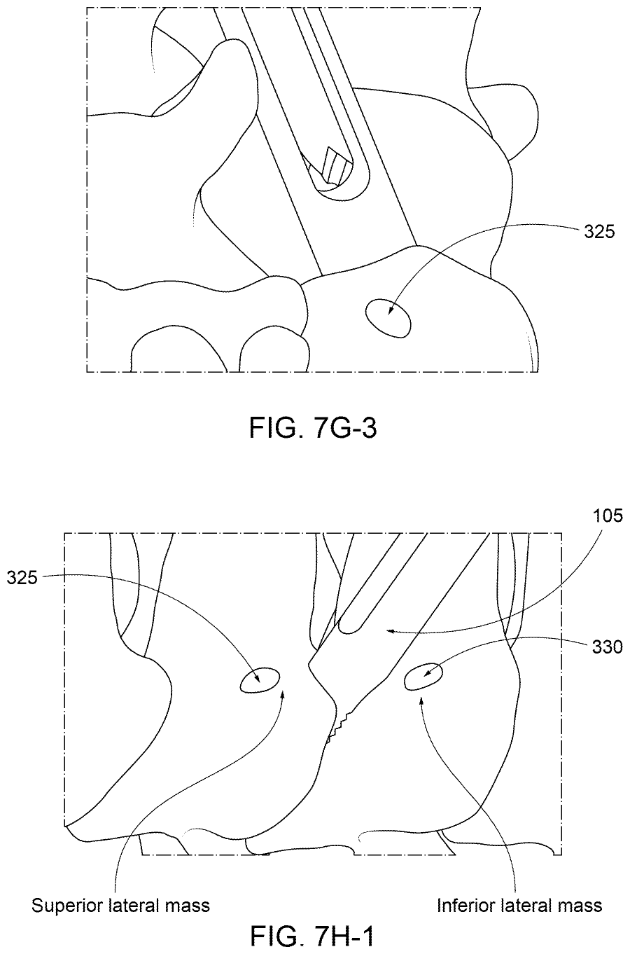

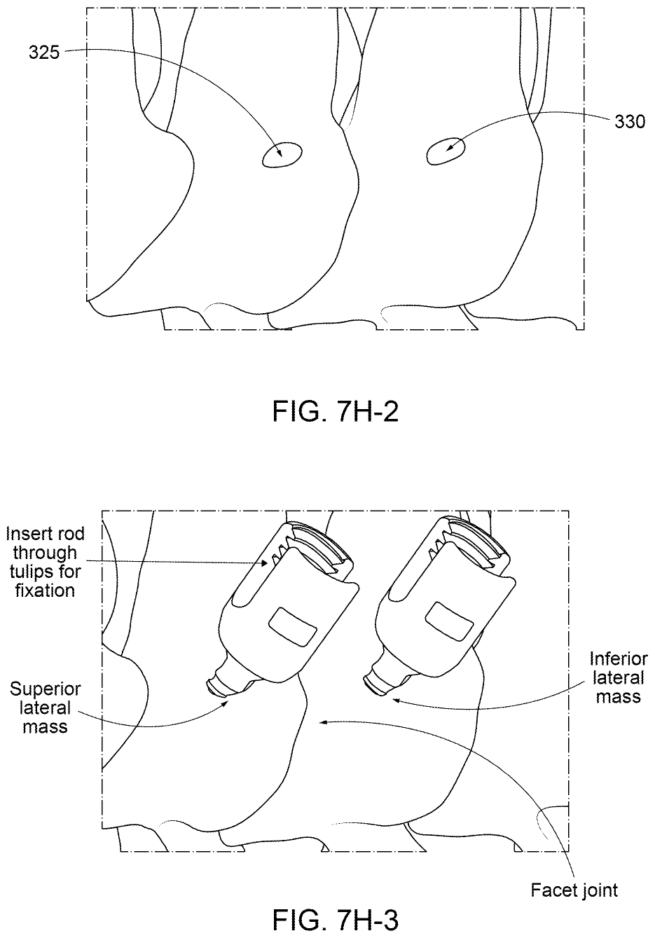

As can be understood from FIGS. 7A-1 through 7A-4, the access chisel 105 is inserted into the facet joint, such as a cervical facet joint, at the desired trajectory. The split point tip 127 of the chisel 105 eases the insertion into the facet joint and the positive stop 130 prevents over insertion. The teeth 135 help to anchor the chisel 105 and to provide some decortication. The window 140 is used to provide visualization of the inferior facet joint. FIG. 7A-3 illustrates a top view after insertion of the chisel showing the correct medial to lateral angulation of the chisel 105. FIG. 7A-4 illustrates how the positive stop 130 has prevented over insertion and how the window 140 can be used to view the inferior lateral mass.

As can be understood from FIGS. 7B-1 through 7B-6, the drill guide 150 is then slid over the access chisel 105 until the tip 185 (serrated teeth) contacts the superior lateral mass. FIGS. 7B-3 through 7B-6 illustrates the drill guide after full insertion over the access chisel such that the tip (serrated teeth) is abutting the lateral mass. The surgeon can position the drill guide by rotating the drill guide about the access chisel. That is, because the drill guide 150 can freely rotate about the access chisel 105, the surgeon can position the tip at a precise or desired location on the lateral mass. FIG. 7B-4 illustrates the free rotation of the drill guide 150 where the medial positioning of the tip 185 can be rotated counterclockwise relative to the chisel 105 and FIG. 7B-5 shows the lateral positioning of the tip 185 with a clockwise rotation. As depicted in FIG. 7B-7, once the final position is chosen, the tip is anchored into position. As can be understood from FIGS. 7B-6 and 7B-8, the surgeon impacts the mallet surface 195 of the drill guide handle to anchor the teeth into cortical bone of the lateral mass. Once anchored, the teeth of the tip prevent the free rotation of the drill guide about the chisel.

As depicted in FIGS. 7C-1 through 7C-4, with the drill guide 150 in place, the drill guide burr 245 is inserted into the second lumen or drill cannula 175 until the tip abuts the lateral mass. With light downward pressure, the burr 245 is rotated clockwise with a manual or power drill to burr 245 a divot 320 in the lateral mass. This divot 320 is the entry point for the drill. The drill guide burr 245 is withdrawn from the cannula 175 (see FIG. 7C-4).

As can be understood from FIGS. 7D-1 through 7D-2, the drill 280 is then inserted in the drill guide cannula 175 until the tip touches the entry point divot 320 on the lateral mass. With downward pressure, the drill 280 is rotated clockwise with a manual or high speed drill until the positive stop 270 on the drill abuts the tip of the second lumen or drill cannula. In doing this, the drill will drill a hole 325 to the set depth (see reference discussion with respect to FIGS. 4F-1 through 4G-3). FIG. 7D-1 and 7D-2 illustrates the drill being inserted into the second lumen or drill cannula 175. FIGS. 7E-1 through 7E-3 illustrate the drill tip 275 positioned in the burr divot (FIG. 7E-1), the drill engaged in the bone (FIG. 7E-2) and then the final hole after the drill is removed (FIG. 7E-3). FIGS. 7F-1 through 7F-3, and with reference to FIGS. 4F-1 through 4F-3 and related text, illustrate the optional drill depths that may be achieved.

As can be understood from FIGS. 7G-1 through 7G-3, once the hole 325 is drilled in the superior lateral mass, the drill guide 150 is rotated 180 degrees about the access chisel to drill the inferior lateral mass using the steps as described above. As described, the window 140 of the access chisel 105 is used to directly visualize the inferior lateral mass during tip placement (FIG. 7G-1), burring (FIG. 7G-2) and drilling (FIG. 7G-3).

FIG. 7G-1 illustrates placement of the visualization window 140 showing the drill guide tip 275 placement, then the burring step (FIG. 7G-2) and then the drilling step (FIG. 7G-3). FIGS. depict a side view showing a superior and inferior lateral mass holes 325, 330 after they have been drilled with the access chisel in place (FIG. 7H-1) and then with the access chisel removed (FIG. 7H-2).

FIG. 7H-3 depicts placement of a fixation system (screws and tulips, which ultimately receive a rod for fixation) in the holes 325, 330 drilled by the drill guide system 100 described herein.

Of course, it is to be appreciated that any one of the examples, embodiments or processes described herein may be combined with one or more other examples, embodiments and/or processes or be separated and/or performed amongst separate devices or device portions in accordance with the present systems, devices and methods.

Finally, the above-discussion is intended to be merely illustrative of the present system and should not be construed as limiting the appended claims to any particular embodiment or group of embodiments. Thus, while the present system has been described in particular detail with reference to exemplary embodiments, it should also be appreciated that numerous modifications and alternative embodiments may be devised by those having ordinary skill in the art without departing from the broader and intended spirit and scope of the present system as set forth in the claims that follow. Accordingly, the specification and drawings are to be regarded in an illustrative manner and are not intended to limit the scope of the appended claims.

1. A drill guide system comprising:
an access chisel; and
a drill guide comprising:
a cannulated body portion comprising:
a first lumen comprising a first central longitudinal axis, and
a second lumen comprising a second central longitudinal axis;
a proximal portion comprising:
a handle portion comprising:
a first through hole to provide access to the first lumen,
a second through hole to provide access to the second lumen, and
a depth stop mechanism comprising:
one or more slots defined in the handle portion, and
a depth stop bushing comprising a tubular body, a knob and
a shaft connecting the knob and the tubular body, the shaft having
a shaft protrusion extending a length of the shaft;
wherein the tubular body of the depth stop bushing is received in the second lumen.

2. The drill guide system of claim 1, wherein each of the one or more slots comprises a bottom portion and a slot protrusion extending from a side proximate to a top of the bottom portion, the slot protrusion configured to extend or retract when pressed with the shaft protrusion to lock or release the depth stop bushing in a respective slot.

3. The drill guide system of claim 2, wherein each of the one or more slots is U-shaped.

4. The drill guide system of claim 1, wherein said first and second central longitudinal axes are distinct and parallel.

5. The drill guide system of claim 1, wherein the handle portion further comprises a mallet surface extending from a side of the handle portion

6. The drill guide system of claim 1, wherein the first lumen is configured to receive the access chisel and the second lumen is configured to receive a drill.

7. The drill guide system of claim 1, wherein a distal portion of the second lumen further comprises serrated teeth configured for anchoring the drill guide into the cortical bone of the lateral mass.

8. The drill guide system of claim 1, further comprising a ring feature configured to join the two lumens together proximate the distal portion of the drill guide.

9. The drill guide system of claim 1, wherein the access chisel is a facet access chisel and the drill guide is a lateral mass drill guide.

10. The drill guide system of claim 1, further comprising a drill guide burr, the drill guide burr comprising an elongated body with a proximal portion opposite a distal portion, the proximal portion comprising a positive stop feature and a quick connect feature configured to receive a handle, and the distal portion comprising a rounded cross-cut burr tip configured to create a divot in a lateral mass.

11. The drill guide system of claim 10, wherein the elongated body has a length and the proximal portion has a first diameter and the distal portion has a second diameter and the first diameter is greater than the second diameter

12. The drill guide system of claim 1, further comprising a drill, the drill comprising an elongated body with a proximal portion opposite a distal portion, the proximal portion comprising a positive stop feature and an a/o quick connect feature configured to receive a handle, and the distal portion comprising a short flute drill profile and an awl tip

13. The drill guide system of claim 12, wherein the elongated body has a length and the proximal portion has a first diameter and the distal portion has a second diameter and the first diameter is greater than the second diameter.

14. The drill guide system of claim 1, wherein the access chisel comprises:
an elongated body having a proximal portion and a distal portion;
a chamfered or beveled end feature positioned at the distal portion of the body and configured for insertion at the facet joint;
an window defined in the distal portion; and
a stop feature defined in the distal portion and configured to abut a posterior edge or portion of a facet joint.

15. The drill guide system of claim 14, wherein the distal portion of the access chisel further comprises a raised or protruding feature.

16. The drill guide system of claim 15, wherein the raised or protruding feature is a plurality of spikes or teeth.

17. (canceled)

18. The drill guide system of claim 14, wherein the proximal portion has a first height and the distal portion has a second height and the first height is greater than the second height.

19. The drill guide system of claim 9, wherein the facet joint is a cervical facet joint.

20.-33. (canceled)
